(19) United States
(12) Patent Application Publication
(10) Pub. No.: US 2026/0363528 A1
(43) Pub. Date: Nov. 19, 2026

(54) ACTUATORS FOR MEDICAL DEVICES AND RELATED SYSTEMS AND METHODS

(71) Applicants: Boston Scientific Scimed, Inc., Maple Grove, MN (US); Boston Scientific Medical Device Limited, Galway (IE)

(72) Inventors: Aditya DHANOTIYA, Pune (IN); Nabarun BHOWMICK, Kolkata (IN); Shrikant Vasant RAUT, Pune (IN); Balaji ASWATHA NARAYANA, Bangalore (IN); James WELDON, Newton, MA (US)

(73) Assignees: Boston Scientific Scimed, Inc., Maple Grove, MN (US); Boston Scientific Medical Device Limited, Galway (IE)

(21) Appl. No.: 19/755,296

(22) Filed: Jul. 27, 2026

Related U.S. Application Data
(63) Continuation of application No. 18/303,263, filed on Apr. 19, 2023.
(60) Provisional application No. 63/363,253, filed on Apr. 20, 2022.

(51) Int. Cl. A61B 1/00

(52) U.S. Cl. CPC A61B 1/00066; A61B 1/00098

(57) ABSTRACT

A handle assembly for a medical device including a handle body, and an actuator for controlling an elevator at a distal portion of the medical device, the actuator including a contact portion including a first portion positioned exterior to the handle body and a second portion positioned within an interior portion of the handle body, and an interior body coupled to (i) the contact portion and (ii) a shaft within the handle body, wherein the actuator is configured to move across an exterior surface of the handle body between a first position and a second position, and the actuator is configured to move the elevator to a third position when the user moves the actuator to the first position, and move the elevator to a fourth position when the user moves the actuator to the second position.

### CROSS-REFERENCE TO RELATED APPLICATIONS

This application is a continuation of U.S. patent application Ser. No. 18/303,263, filed on Apr. 19, 2023, which claims the benefit of priority under 35 U.S.C. § 119 from U.S. Provisional Application No. 63/363,253, filed Apr. 20, 2022, each of which is incorporated by reference herein in its entirety.

### TECHNICAL FIELD

Various aspects of this disclosure relate generally to actuators and handle assemblies of medical devices. More specifically, embodiments of this disclosure relate to ergonomic actuators and handle assemblies for an endoscope or other medical device, among other aspects.

### BACKGROUND

During endoscopic procedures, the medical professional operating the endoscope often wraps his/her entire palm around a grip or handle portion of the device. Various actuators on the handle of the endoscope require the medical professional to contort his/her hands frequently and for prolonged periods of time during a procedure, which can cause strain, or even an injury. In some cases, actuation of different scope controls, like knobs or an elevator, may result in excessive movements of the medical professional's thumb or other fingers, which may result in strain in the medical professional's hand. Endoscope operators can experience wrist and hand discomfort resulting from holding and manipulating the endoscope's handle. In some cases, medical professionals may experience symptoms similar to those of Carpal Tunnel Syndrome, tendonitis, or De Quervain's tenosynovitis. When a medical professional experiences fatigue or other pain in the fingers, hand, or wrist, the medical professional may shift from a primary grip position to a secondary grip position that may be a less powerful grip than the primary grip position, such as shifting from a four finger grip to a three finger grip. Repeatedly reaching or contorting the fingers to access various actuators can increase fatigue or other pain.

When a medical professional repeatedly readjusts his or her hand grip in between procedure tasks, the procedure may be prolonged and procedural tasks may be more difficult. Depending on the size of a medical professional's hand, actuators may be positioned in non-optimized positions and increase the number of readjustments of the professional's hand during a procedure.

### SUMMARY

According to an example, a handle assembly for a medical device may comprise a handle body and an actuator for controlling movement of an actuation wire within a medical device, the actuator may comprise a contact portion including a first portion positioned exterior to the handle body and a second portion positioned within an interior portion of the handle body, and an interior body coupled to (i) the contact portion and (ii) a shaft within the handle body, wherein the actuator may be configured to move across an exterior surface of the handle body between a first position and a second position, and the actuator may be configured to move the actuation wire to a third position when the user moves the actuator to the first position, and move the actuation wire to a fourth position when the user moves the actuator to the second position.

In another example, the interior body may be an arm that extends radially outward from a central longitudinal axis of the shaft and configured to rotate the shaft. A ring may be rotatably coupled to the shaft via couplings that extend radially outward from a central longitudinal axis of the shaft, and wherein a rod may be rotatably coupled to the ring at a first end and to the actuation wire at a second end. The actuation wire may be coupled to an elevator at a distal end of the medical device. The shaft may be rotatable relative to the handle body and longitudinally extends in a direction transverse to the longitudinal axis of the medical device. The arm may include an opening configured to receive a portion of the second portion of the contact portion. The contact portion may be configured to move along a curved path. The handle body may include a slot extending longitudinally within a recessed portion of the exterior surface of the handle body. The slot may include an opening on a first side of the handle body and a second side of the handle body, wherein the first side may be on an opposite side from the second side. The contact portion may be positioned between a camera button and at least one knob actuator. The interior body may include a curved gear positioned within the interior of the handle body, wherein the curved gear may be moveable along a curved channel within the interior of the handle body. External teeth of the curved gear may contact external teeth of an adjacent gear to drive rotation of the adjacent gear and the shaft. A ring may be coupled to the shaft via couplings extending radially outward from the shaft, and wherein a rod may be rotatably coupled to the ring and the actuation wire. The second portion of the contact portion may include first external teeth configured to contact second external teeth of an adjacent first gear positioned inside the handle body to drive rotation of the shaft. Rotation of the first gear may drive rotation of an adjacent second gear.

According to an example, a handle assembly for a medical device may comprise a handle body and an actuator for controlling one or more components at a distal portion of the medical device, the actuator may comprise a contact portion including a first portion positioned exterior to the handle body and a second portion positioned within an interior portion of the handle body, and an interior body coupled to (i) the contact portion and (ii) a shaft within the handle body, wherein the actuator may be configured to move across an exterior surface of the handle body to actuate one or more components at the distal portion of the medical device. A proximal portion of the handle body may include an opening extending in an arc on the exterior surface of the handle body, wherein the opening is configured to receive the actuator. The one or more components may be an elevator.

According to an example, a method of moving an elevator at a distal portion of an endoscopic device, the endoscopic device may comprise a handle body, an actuator including a contact portion including a first portion positioned exterior to the handle body and a second portion positioned within an interior portion of the handle body, and an interior body coupled to (i) the contact portion and (ii) a shaft within the handle body, the method may comprise moving the actuator across an exterior surface of the handle body to a first position to move the elevator to a second position, and moving the actuator across an exterior surface of the handle body to a third position to move the elevator to a fourth position. The method may further comprise moving the first portion of the contact portion along a linear path.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIGS. 1A and 1B are perspective views of an exemplary endoscope, according to aspects of this disclosure.

FIGS. 2A and 2B are perspective views of a user's hand holding an endoscope handle, according to aspects of this disclosure.

FIGS. 3A and 3B are perspective views of a portion of an endoscope handle assembly, according to aspects of this disclosure.

FIG. 3C is a perspective view of an interior portion of an endoscope handle assembly, according to aspects of this disclosure.

FIG. 4A is a perspective view of a portion of an endoscope handle assembly, according to aspects of this disclosure.

FIG. 4B is a perspective view of a user's hand holding the endoscope handle of FIG. 4A, according to aspects of this disclosure.

FIG. 4C is a perspective view of an endoscope handle assembly, according to aspects of this disclosure.

FIGS. 4D and 4E are exploded views of an exemplary elevator actuator, according to aspects of this disclosure.

FIGS. 5A-5C are various perspective views of an endoscope handle assembly, according to aspects of this disclosure.

FIG. 5D is a cross-sectional view of an endoscope handle assembly, according to aspects of this disclosure.

FIG. 5E is a perspective view of a portion of an exemplary actuation mechanism, according to aspects of this disclosure.

FIG. 6 is a cross-sectional view of an endoscope handle assembly, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the patient. Throughout the figures included in this application, arrows labeled “P” and “D” are used to show the proximal and distal directions in the figure. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

Embodiments of this disclosure seek to improve a user's ability to grip, manipulate, and otherwise apply force to a handle, and actuators of the handle, of a medical device, such as an endoscope, during a medical procedure. Embodiments of this disclosure may help reduce the need to reposition a user's hand during a procedure, reduce strain to a user's hand from excessive movement of fingers and/or uncomfortable positioning of fingers, and maximize muscle strength during operation of a medical device, among other aspects.

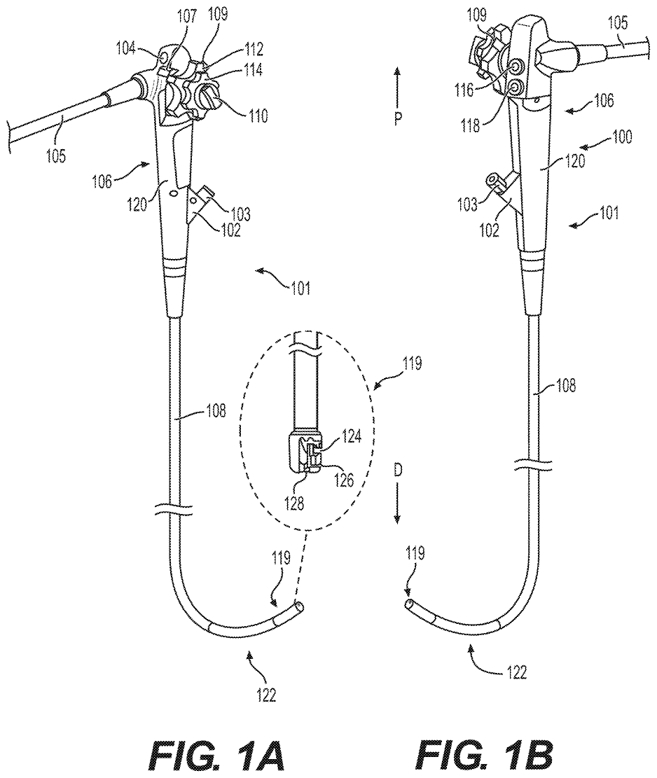

FIGS. 1A and 1B show a perspective view of an exemplary endoscope system 100 including endoscope 101. The endoscope 101 may include a handle assembly 106 and a flexible tubular shaft 108. The handle assembly 106 may include a biopsy port 102, a biopsy cap 103, an image capture button 104, an elevator actuator 107, a first locking lever 109, a second locking lever 110, a first control knob 112, a second control knob 114, a suction button 116, an air/water button 118, a handle body 120, and an umbilicus 105. All of the actuators, elevators, knobs, buttons, levers, ports, or caps of the endoscope system 100 may serve any purpose and are not limited by any particular use that may be implied by the respective naming of each component used herein. The umbilicus 105 may extend from the handle body 120 to auxiliary devices, such as a control unit, water supply, or vacuum source. The umbilicus 105 therefore can transmit signals between the endoscope 101 and the control unit, to control lighting and imaging components of the endoscope 101 and/or receive image data from the endoscope 101. The umbilicus 105 also can provide fluid for irrigation from the water supply and/or suction to a distal tip 119 of the shaft 108. The buttons 116 and 118 control valves for suction and fluid supply, respectively. The shaft 108 may terminate at the distal tip 119. The shaft 108 may include an articulation section 122 for deflecting the distal tip 119 in up, down, left, and/or right directions. The knobs 112 and 114 may be used for controlling such deflection, and the locking levers 109 and 110 may lock the knobs 112 and 114, respectively, in desired positions. The handle body 120 may be tapered and may narrow as the handle extends distally such that the profile of the handle body 120 is smaller at its distal end than at its proximal end.

The distal tip 119 may include an imaging device 124 (e.g., a camera) and a lighting source 126 (e.g., an LED or an optical fiber). The distal tip 119 may be side-facing. That is, imaging device and lighting source may face radially outward, perpendicularly, approximately perpendicularly, or otherwise transverse to a longitudinal axis of the shaft 108 and the distal tip 119.

The distal tip 119 may also include an elevator 128 for changing an orientation of a tool inserted in a working channel of the endoscope 101. The elevator 128 may alternatively be referred to as a swing stand, pivot stand, raising base, or any suitable other term. The elevator 128 may be pivotable via, e.g., an actuation wire or another control element that extends from the elevator actuator 107 on handle assembly 106, through the shaft 108 to the elevator 128.

Although the term endoscope may be used herein, it will be appreciated that other devices, including, but not limited to, duodenoscopes, colonoscopes, ureteroscopes, bronchoscopes, laparoscopes, sheaths, catheters, or any other suitable delivery device or medical device may be used in connection with the devices of this disclosure.

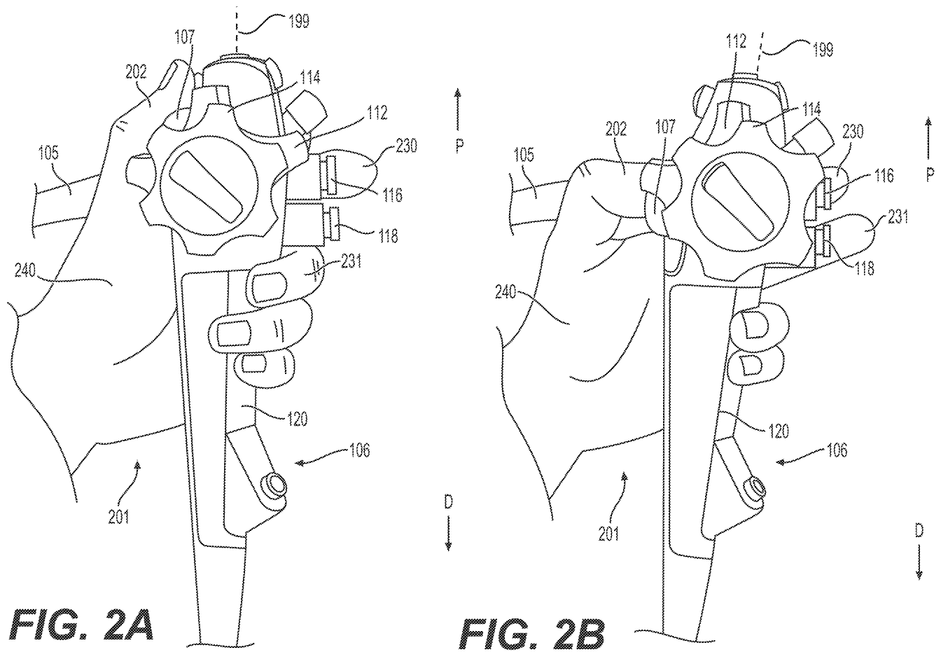

In operating the endoscope system 100, a user may use his/her left hand to hold the handle assembly 106 (shown in FIG. 2A) while the right hand is used to hold accessory devices and/or operate one or more of the actuators of the handle assembly 106, such as the first and second control knobs 112, 114 and the first and second locking levers 109, 110. The user may grasp the handle assembly 106 by wrapping the user's hand around the handle body 120. When grasping handle body 120, the user may use the left thumb to operate the first and second control knobs 112, 114 and the elevator actuator 107 (through rotation about their axis), and may use a left-hand finger to operate the image capture button 104, the suction button 116, and the air/water button 118 (each by pressing).

FIGS. 2A and 2B show an exemplary user's left hand 201 grasping the handle assembly 106. The user's left index finger 230 and middle finger 231 may be used to operate the suction button 116 and the air/water button 118. A user may have to reach and/or strain their index or middle finger to actuate the suction button 116 and/or the air water button 118. A user may have to reach and/or strain their left thumb and/or hold their left thumb in uncomfortable positions to operate the first and second control knobs 112, 114, and/or the elevator actuator 107. The actuators and handle assemblies discussed herein below may help reduce the reach required and/or alleviate strain to a user's fingers and/or hand. The handle assembly 106 may have a central longitudinal axis 199 extending longitudinally through the handle assembly 106.

The user may position the thumb 202 of the grasping hand 201 over the elevator actuator 107 and move the elevator actuator 107 along a circular path from a first position (shown in FIG. 2A) to a second position (shown in FIG. 2B) by moving the thumb 202. As shown in FIG. 2B, the palm 240 may move away from the handle body 120 when the thumb 202 moves from the first position to the second position, and a user may have difficulty reaching the suction button 116 and/or the air water button 118 with the index or middle fingers when the palm 240 moves away from the handle body 120. The actuators and handle assemblies discussed herein below may help reduce the movement required of the index and/or ring fingers to actuate the suction button 116 and/or the air water button 118, and may reduce the movement required of the thumb to actuate an elevator actuator 107 or other actuator during operation of the endoscope 101.

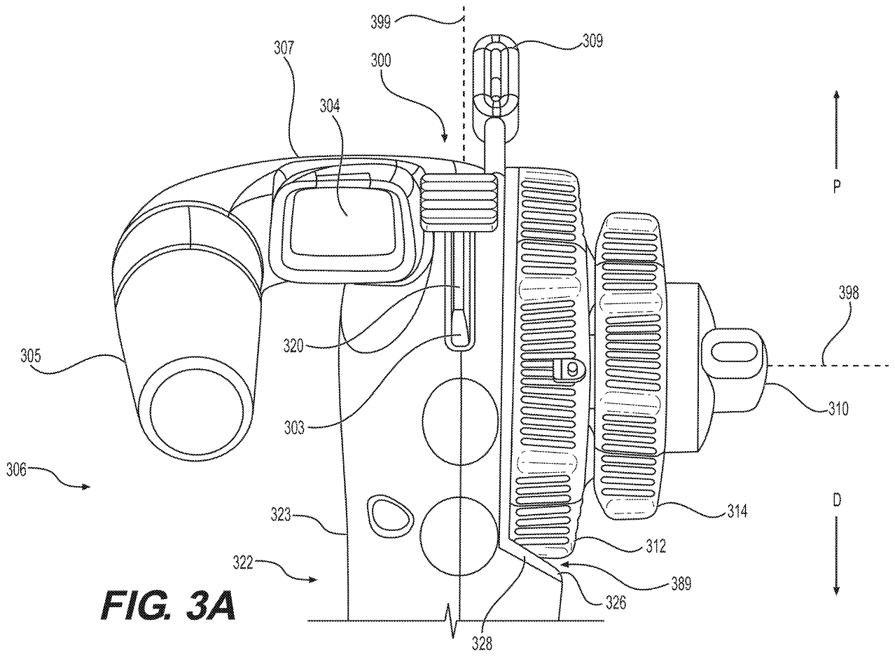

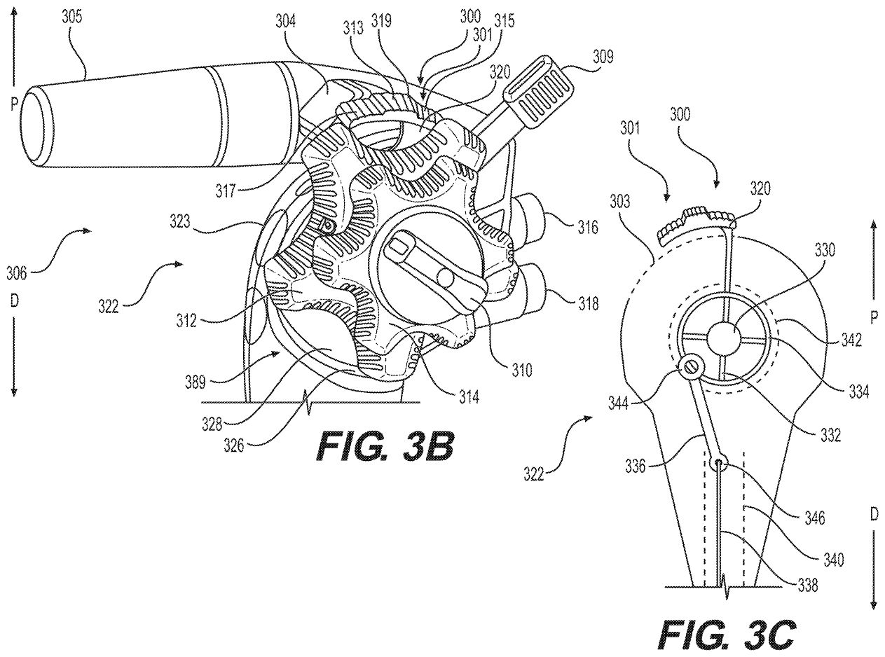

FIGS. 3A and 3B show perspective front and side views of a proximal end of an alternative handle assembly 306 including a handle body 322, an image capture button 304, a first locking lever 309, a second locking lever 310, a first control knob 312, a second control knob 314, a suction button 316, an air/water button 318, an umbilicus 305, and an elevator actuator 300. The handle body 322, at its proximal end, may have a circular head portion 323. The remainder of the handle body 322 may extend longitudinally and may narrow as the handle body 322 extends distally. In some examples, the head portion 323 may include approximately the proximal half of the handle body 322 from an approximate center of the handle body 322 to a proximalmost end of the handle body 322. A central rotational knob axis 398 may extend through the first control knob 312 and the second control knob 314. A central longitudinal axis 399 extends longitudinally through the handle assembly 306. A slot 303 is positioned towards the top of the head portion 323 of the handle body 322. The slot 303 is configured to receive a portion of the elevator actuator 300 and permits movement of the elevator actuator 300 along a circular path. The slot 303 may extend in an arc starting from a point located at the top of the head portion 323 of the handle body 322 and ending at a point on or above an axis parallel to the central rotational knob axis 398, although other arc lengths are also contemplated. The image capture button 304 may project outwardly from a curved portion 307 of the handle body 322 leading to the umbilicus 305, may be positioned between the umbilicus 305 and the elevator actuator 300, and may be spaced from the central longitudinal axis 399. The image capture button 304 may have a smooth, flat surface that may allow the user's thumb 202 to easily access the image capture button 304. The image capture button 304 may have a length of 15 millimeters and a width of 10 millimeters.

The head portion 323 includes a recess 389 on the right side of the handle body 322, and the recess 389 may be defined by a first circular surface 328 that intersects with a curved surface 326. The recess 389 is configured to receive the control knobs 312 and 314. The curved surface 326 may be transverse to the surface 328. The curved surface 326 is positioned at a first lateral side of the head portion 323 and at an opposite side from the umbilicus 305. The first control knob 312 is positioned within the recess 389 so that an outer surface of the first control knob 312 is in line with or substantially in line with the edge of the curved surface 326 and the second control knob 314 is positioned outside of the recess 389. The recess 389 allows the control knobs 312 and 314 to be positioned closer to the central longitudinal axis 399, which reduces the distance between the various actuators of the handle assembly 306, such as the distance between the first control knob 312 and the elevator actuator 300. By reducing the distance between the various actuators of the handle assembly 306, a user may more easily access the various actuators, such as the first control knob 312, the second control knob 314, and the camera button 304, with a single hand and may reduce the amount of reach of a user's fingers required to actuate each of the various actuators of the handle assembly 306. For example, the control knobs 312 and 314 may be shifted ten millimeters closer to the central longitudinal axis 399 compared to if the handle body 322 did not include the recess 389. This may allow the user's thumb 202, and especially a thumb from a user with smaller hands, to easily reach/access the control knobs 312 and 314. In addition, the positioning of the control knobs 312 and 314 relative to the central longitudinal axis 399 reduces the degree of abduction required of the thumb to reach the control knobs 312 and 314 and thus may allow for a user to utilize maximum effort of their muscle to rotate the control knobs 312 and 314.

As shown in FIG. 3B, a first position of the elevator actuator 300 within the slot 303 may be towards the top of the head portion 323. The elevator actuator 300 may include a contact portion 301. The contact portion 301 may include a center portion 313, and a top portion 315 and a bottom portion 317 recessed from the center portion 313. The center portion 313 protrudes outwardly, further from the handle body 322, relative to the portions 315 and 317. The top portion 315 and the bottom portion 317 may extend in opposite directions from opposite sides of the center portion 313. The contact portion 301 may be substantially rectangular, and may be curved so that it is convex towards a user of the endoscope. The portions 315 and 317, and positioning of the elevator actuator 300 towards the proximal end of the handle body 322 may allow the user's thumb 202 to easily access and operate the extended elevator actuator 300.

The outer surface of the center portion 313, the top portion 315, and the bottom portion 317 may include grip projections 319. Each grip projection 319 protrudes from adjacent portions of the outer surface having the grip projection 319. Each grip projection 319 may extend from side-to-side, transverse, or substantially perpendicular, to the proximal/distal directions. The use of “top” or “upper” and “bottom” or “lower,” or like terms, as modifiers for structure in embodiments of this disclosure refers to the relative position of that structure when an endoscope is in a typical position during use, such as the positions shown in the Figures.

The elevator actuator 300 may further include a connector 320 coupled to the contact portion 301 at one end and coupled to structure internal to the handle body 322 at the other end, for controlling one or more aspects of the endoscope 101, such as pivoting of the elevator 128. For example, as shown in FIG. 3C, the connector 320 may extend from an outer surface of the contact portion 301 and within the handle body 322 to a rotatable shaft 330. The shaft 330 may be coupled to a ring 334 through couplings 332 that extend radially outward from the shaft 330 to the ring 334. A rod 336 may be rotatably coupled to the ring 334 at a first end 344, and rotatably coupled to an actuation wire 338 at a second end 346 for actuation of one or more components of the device, such as the elevator 128. The actuation wire 338 may be moveable within a tubular member 340 within the handle body 322. In some examples, the user's thumb 202 may contact the contact portion 301 and apply a force to move the elevator actuator 300, either proximally or distally, along a circular path formed by the slot 303. Movement of the elevator actuator 300, either proximally or distally, results in the rotation of the shaft 330, and subsequently, the ring 334 via the couplings 332. The first end 344 of the rod 336 may move along a portion of a circular path 342 concentric with the center of the shaft 330 and results in movement of the second end 346 coupled to the actuation wire 338 or any other controlling element to pivot the elevator 128, for example. In some examples, moving contact portion 301 distally may move rod 336 and actuation wire 338 distally, and moving contact portion 301 proximally may move rod 336 and actuation wire 338 proximally. Shaft 330 may be rotatably coupled to an interior portion of handle body 322.

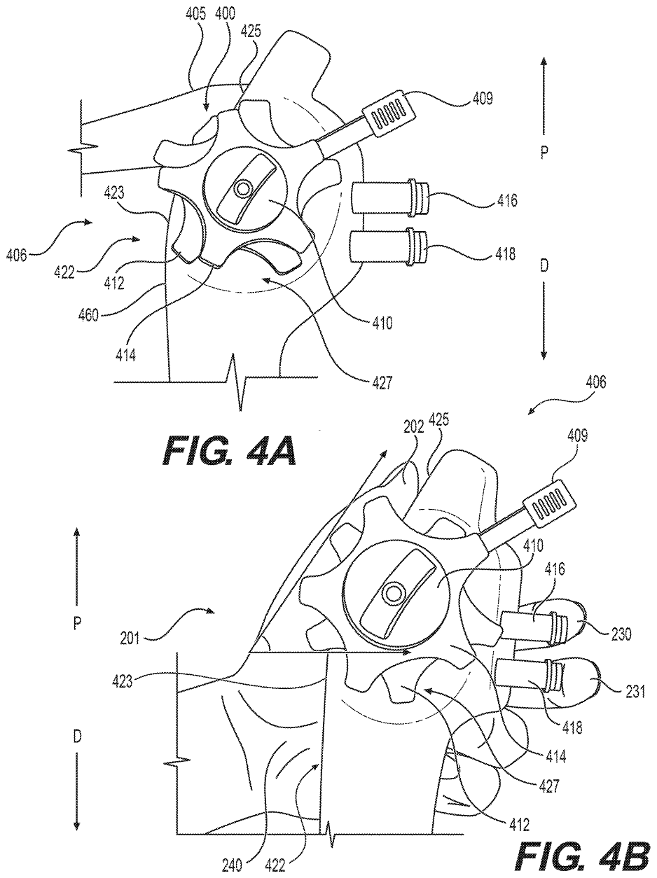

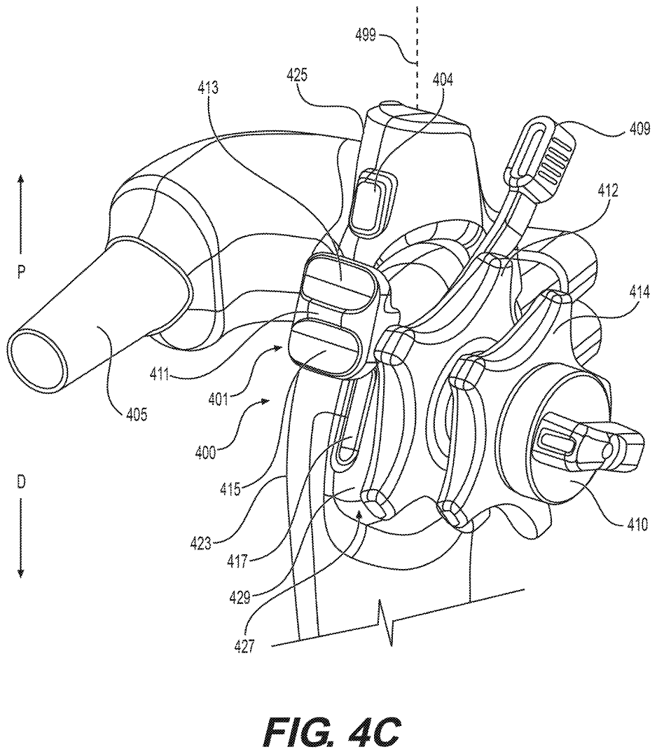

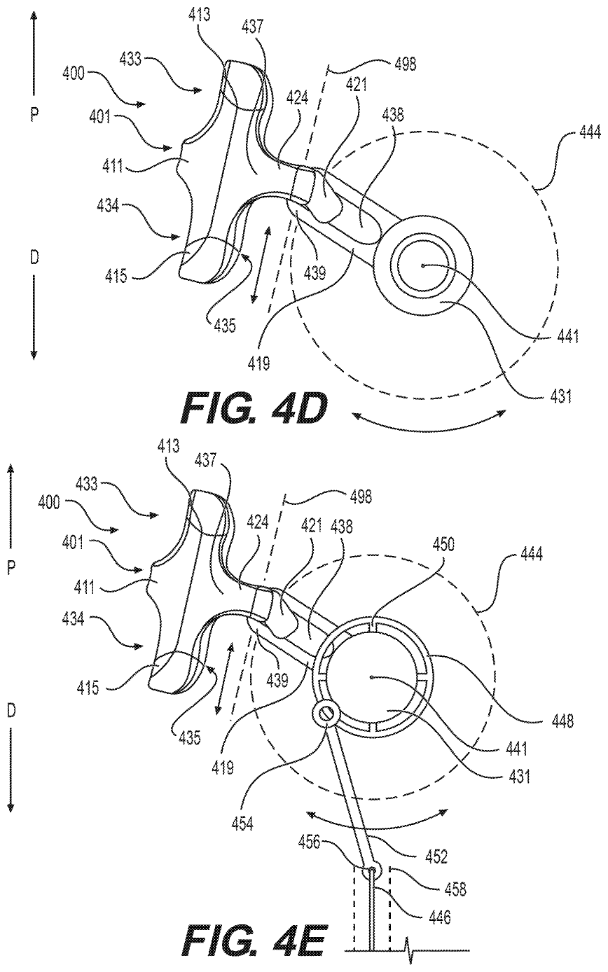

FIGS. 4A-4C show perspective side views of a proximal end of an alternative handle assembly 406 including a handle body 422, an image capture button 404, a first locking lever 409, a second locking lever 410, a first control knob 412, a second control knob 414, a suction button 416, an air/water button 418, an umbilicus 405, and an elevator actuator 400. The handle body 422 includes a head portion 423 at the proximal end of the handle body 422. The head portion 423 may include an angled surface 425 on a front side 460 of the handle body 422. The surface 425 may be angled relative to a forward-facing (i.e. facing the user) outer surface of the handle body 422 and away from the user. As shown in FIG. 4B, the angled surface 425 of the head portion 423 may allow the user's thumb 202 and wrist to be orientated in a natural position during operation of the elevator actuator 400 and the image capture button 404. For example, the user's thumb 202 may be angled at approximately 45 degrees or less when the user is holding the handle assembly 406.

The head portion 423 includes a recess 427 located on the right side of the handle body 422 and is configured to receive the control knobs 412 and 414. The recess 427 includes a base 429 that is parallel to or substantially parallel to a central longitudinal axis 499 of the alternative handle assembly 406. The recess 427 allows for the control knobs 412 and 414 to be positioned in close proximity to the central longitudinal axis 499 and may reduce the degree of abduction required by the user's thumb 202 to rotate the control knobs 412 and 414. For example, the degree range of abduction of the user's thumb 202 to rotate control knobs 412 and 414 may be 0 to 90 degrees, which may reduce thumb fatigue during operation of handle assembly 406.

The recess 427 of head portion 423 includes a slot 417 configured to receive a portion of the elevator actuator 400 and permits movement of the elevator actuator 400 along a linear path. The slot 417 may extend longitudinally within the recess 427. As shown in FIGS. 4C-4E, the elevator actuator 400 may include a contact portion 401. The contact portion 401 may include an upper portion 413 that extends inward towards the central longitudinal axis 499 from an elevated central portion 411. The contact portion 401 may include a lower portion 415 that extends inward towards the central longitudinal axis 499 and on the opposite side of the elevated central portion 411 from the upper portion 413. An outer contact surface of the upper portion 413 and an outer contact surface of the lower portion 415 may each curve inward and each transition to substantially planar outer surfaces 433 and 434. The outer surfaces of the elevated central portion 411, the upper portion 413, and the lower portion 415 may include grip projections (not shown) protruding outward away from translation axis 498 to improve gripping capabilities and comfort. A central connecting element 424 (shown in FIG. 4D) protrudes from a bottom 435 of the contact portion 401 and in the opposite direction of the elevated central portion 411. The central connecting element 424 may be U-shaped, and the contact portion 401 and the central connecting element 424 may form a lumen 437. A portion 421 of the central connecting element 424 may extend through a slot 438 of an arm 419 positioned within handle body 422. A first end 439 of the arm 419 may be fixedly coupled to a rotatable shaft 431 such that the arm 419 extends radially-outward from a central longitudinal axis 441 of the shaft 431. The central longitudinal axis 441 of the shaft 431 is shown extending into the page in FIG. 4D. The shaft 431 may be rotatably coupled to a portion of the handle body 422, and may be coupled to one or more actuation wires for actuation of one or more components at the device, such as the elevator 128.

The elevator actuator 400 may be configured to control pivoting of the elevator 128 through a slider-crank mechanism. The user's thumb 202 may be positioned on the contact portion 401 to move the contact portion 401, including the connecting element 424, in a linear direction along the translation axis 498 within the slot 417 of the handle body 422. Movement of the contact portion 401 in a linear direction may allow for the user's thumb 202 to maintain a natural position during operation of elevator 128 and have proper support for the palm 240 to grasp the handle body 422 properly. Movement of the contact portion 401, and subsequently, portion 421 connected to the contact portion 401, either proximally or distally, results in the rotation of the shaft 431 via the arm 419. Shaft 431 may rotate about axis 441, shown extending into the page in FIG. 4E. The shaft 431 may be coupled to a ring 448 through couplings 450 that extend radially outward from the shaft 431 to the ring 448. A rod 452 may be coupled to the ring 448 at a first end 454 and coupled to an actuation wire 446 at a second end 456 for actuation of one or more components of the device, such as the elevator 128. The actuation wire 446 may be moveable within a tubular member 458 within the handle body 422. The portion 421 of the connecting element 424 may move within the slot 417 as a user translates the contact portion 401 along the translation axis 498. The first end 439 of the arm 419 may move along a portion of a circular path 444 concentric with the center of the shaft 431 to rotate the shaft 431, resulting in the rotation of the ring 448. As the ring 448 rotates, the first end 454 of the rod 452 moves along a curved path defined by the ring 448 and the second end 456 of the rod 452 coupled to the actuation wire 446 moves substantially linearly to actuate one or more components of the device. Shaft 431 may be rotatably coupled to an interior portion of handle body 422. In some examples, ring 448 may be spaced from arm 419.

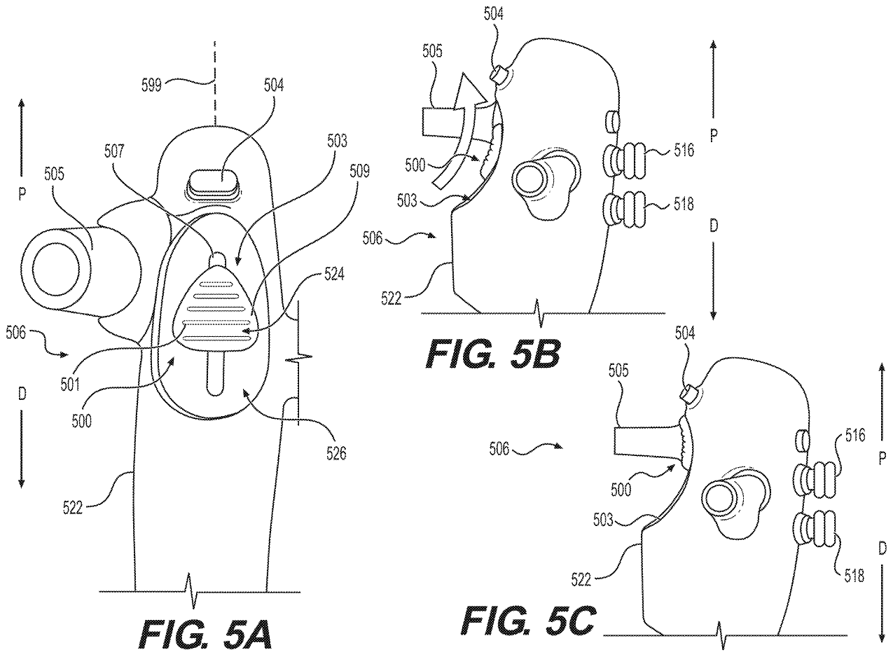

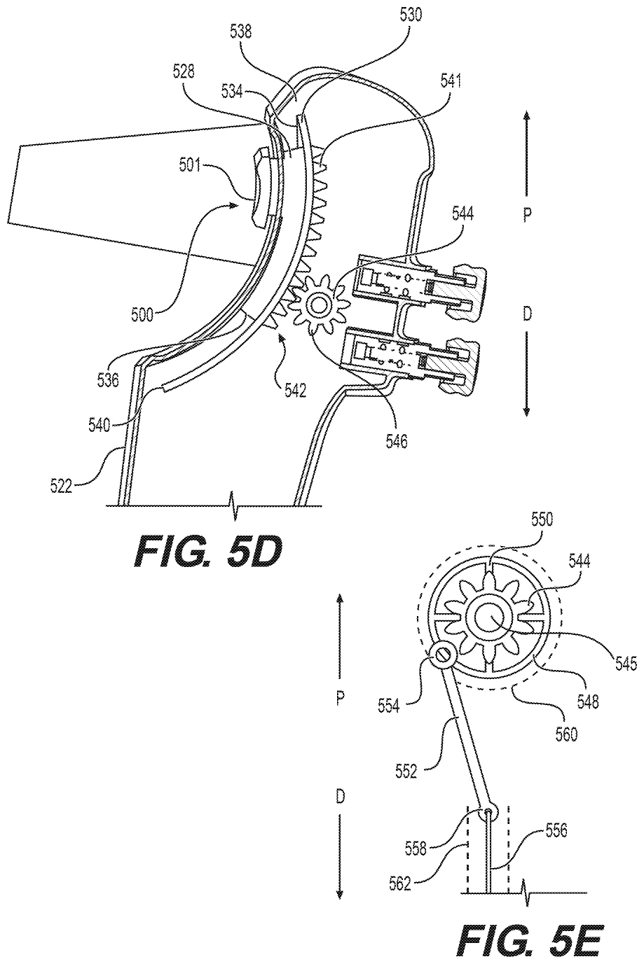

FIGS. 5A-5C show a perspective front and two side views of a proximal end of an alternative handle assembly 506 including a handle body 522, an image capture button 504, a suction button 516, an air/water button 518, an umbilicus 505, and an elevator actuator 500. The handle assembly 506 may further include any of the features described herein in relation to endoscope 101 and/or any of the features of handle assemblies 106, 306, and 406, such as a first control knob, a second control knob, a first locking lever, and a second locking lever (not shown) similar to the first control knob 112, the second control knob 114, the first locking lever 109, and the second locking lever 110 of the endoscope 101. The handle assembly 506 may have a central longitudinal axis 599 extending longitudinally through the alternative handle assembly 506.

A portion 503 of the exterior surface of handle body 522 is concave. The concave portion 503 is positioned below the image capture button 504 and longitudinally aligned with image capture button 504. The concave portion 503 may include a slot 507 that extends longitudinally through a center portion of the concave portion 503, and is configured to receive a portion of the elevator actuator 500. The slot 507 is formed at the center of the concave portion 503 such that the slot 507 is aligned with the central longitudinal axis 599. The slot 507 permits movement of the elevator actuator 500 along a curved path, and elevator actuator 500 is configured to slide across concave portion 503. The elevator actuator 500 includes a contact portion 501 that may be in the shape of a Reuleaux triangle, although other shapes may be contemplated. An outer surface 524 of the contact portion 501 may include grip projections 509 protruding from adjacent portions of the outer surface 524. In one example, the bottom surface of the contact portion 501 may be flush with the outer surface 526 of the concave portion 503 as the contact portion 510 moves along the slot 507.

To operate the elevator actuator 500, the user's thumb 202 may be positioned over the elevator actuator 500 and move the elevator actuator 500 along the curved path from a first position (shown in FIG. 5B) to a second position (shown in FIG. 5C) by moving the thumb 202 proximally. Since the elevator actuator 500 is configured to operate by moving the user's thumb 202 proximally to actuate the elevator 128 (compared to moving the thumb 202 distally), the user may avoid bending of the distal interphalangeal (DIP) joint of the thumb 202 which may alleviate thumb stiffness and avoid a loss of gripping power on the handle body 120. As shown in FIG. 5B, in the first position or initial position, the elevator actuator 500 may be positioned at the a distal end of the slot 507, a center of the slot 507, or any other portion distal from a proximal end of slot 507. As shown in FIG. 5C, the user's thumb 202 may then move the elevator actuator 500 proximally to operate the elevator 128 and, in some examples, actuate elevator 128. When the user desires to hold a position of the elevator 128, the user's thumb 202 is extended to hold the contact portion 501 at a proximal end of the slot 507. Since the thumb 202 is in an extended position compared to a contracted or bent position, the user may more easily maintain gripping power on the handle body 522 and reduce fatigue of the thumb 202 during operation of elevator actuator 500. In some examples, elevator actuator 500 may have a spring bias towards a specific position, such as a position in which the elevator is in an extended or retracted position.

In one example, the elevator actuator 500 may be configured to control pivoting of the elevator 128 through a rack and pinion mechanism. As shown in FIG. 5D, the contact portion 501 may be coupled to a curved gear 528 that extends longitudinally from a first end 534 to a second end 536. The second end 532 is positioned distal from and spaced from a distal end of the contact portion 501. Gear 528 is positioned inside the handle body 522, such that as the contact portion 501 moves proximally or distally, so does a portion of the gear 528 within a channel 530. For example, as a user's thumb 202 moves the contact portion 501 distally along the curved path of the slot 507, the gear 528 moves along a curved path of the channel 530. The channel 530 may extend longitudinally inside of the handle body 522 from a first end 538 to a second end 540, and the second end 540 may be positioned distal from the distal end of the slot 507. The channel 530 is configured to receive gear teeth 546 that project from a lateral side 542 of the linear gear 528. As the linear gear 528 moves proximally or distally along the curved path of the channel 530, the external teeth 541 contact gear teeth 546 of an adjacent gear 544 positioned within the handle body 522 to drive rotation of the gear 544. Gear 544 may be coupled to a rotatable shaft 545 within handle body 522. As shown in FIG. 5E, the gear 544 and shaft 545 may be coupled to a ring 548 through couplings 550 that extend radially outward from the shaft 545 to ring 548. A rod 552 may be coupled to the ring 548 at a first end 554 and coupled to an actuation wire 556 at a second end 558 for actuation of one or more components of the device, such as the elevator 128. The actuation wire 556 may be moveable within a tubular member 562 positioned within the handle body 522. The first end 554 of the rod 552 may move along a circular path 560 concentric with the center of the gear 544 and results in movement of the second end 558 coupled to the actuation wire 556 to pivot elevator 128, for example.

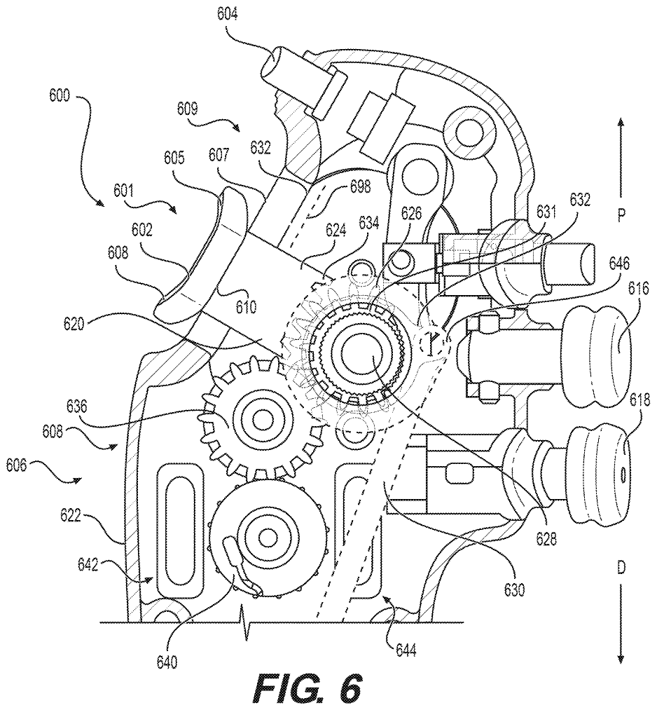

FIG. 6 shows a cross-sectional view of a proximal end of an alternative handle assembly 606 including a handle body 622, an image capture button 604, a suction button 616, an air/water button 618, and an elevator actuator 600. The handle assembly 606 may further include any of the features of the handle assemblies 106, 306, 406, and 506, such as an umbilicus, a first control knob, a second control knob, a first locking lever, and a second locking lever (not shown) similar to the umbilicus 105, the first control knob 112, the second control knob 114, the first locking lever 109, and the second locking lever 110 of the endoscope 101.

A portion 609 of the exterior surface of the handle body 622 may be recessed. The recessed portion 609 of the handle body 622 includes a slot 607 configured to receive a portion of the elevator actuator 600, permits movement of the elevator actuator 600 in the proximal and distal directions, and restrains movement of the elevator actuator 600 in lateral directions. The slot 607 may extend longitudinally within the recessed portion 609, and a longitudinal axis 698 of slot 607 may be transverse to a central longitudinal axis of handle body 622. The elevator actuator 600 may include a contact portion 601. Contact portion 601 may include a planar central portion 602, a top portion 605, and a bottom portion 608. The top portion 605 and the bottom portion 608 may extend in opposite directions from opposite sides of the central portion 602 and curve away from the handle body 622. In another example, the contact portion 601 may be similar to the contact portion 501. A central connecting element 620 extends from a bottom surface 610 of the contact portion 601 to a position inside of the handle body 622 adjacent to a first gear 626. Connecting element 620 may be moveable within the slot 607 along axis 698. Connecting element 620 may be any suitable shape that secures connecting element 620 within slot 607 and permits movement of connecting element 620 in the proximal and distal directions. For example, connecting element 620 may be I-shaped. A portion 624 of the connecting element 620 is positioned inside the handle body 622.

Connecting element 620 may include gear teeth 634 at a proximal portion of connecting element 620, positioned at an end opposite from the end of connecting element 620 coupled to contact portion 601. Gear teeth 634 may be engaged with a first gear 626 within handle body 622. First gear 626 may be fixedly coupled to a shaft 628, and shaft 628 may be rotatable coupled to handle body 622 within handle body 622. A second gear 631 may also be coupled to shaft 628, adjacent to first gear 626, and may be engaged with a third gear 636 within handle body 622. A ring 632 (shown in dotted lines) may also be coupled to shaft 628, and ring 632 may be rotatably coupled to rod 630 (shown in dotted lines). Ring 632 and rod 630 may have any of the features of ring 548 and rod 552 discussed hereinabove. Translation of contact portion may move 601 gear teeth 634, which rotates first gear 626 and shaft 628, and causes ring 632 to also rotate via shaft 628 to translate rod 630 proximally or distally.

In one example, the user's thumb 202 may be positioned on the contact portion 601 to move the contact portion 601, including the connecting element 620, in a linear direction along the translation axis 698. As the connecting element 620 moves proximally or distally along the translation axis 698, gear teeth 634 contact teeth of first gear 626, resulting in rotation of the first gear 626 and second gear 631 in a first direction (e.g., clockwise or counter-clockwise). The teeth of second gear 631 contact teeth of third gear 636 resulting in rotation of the third gear 636 in a second direction (e. g, clockwise or counter-clockwise). As first gear 626 and second gear 631 rotate in the first direction, shaft 628 and ring 632 also rotates in the first direction to move rod 630 in a proximal or distal direction. A first end 646 of rod 630 may be coupled to the ring 632, and first end 646 rotates about shaft 628 to move rod 630 in a proximal or distal direction.

The handle assemblies 306, 406, 506, 606 and the actuators 300, 400, 500, 600 of this disclosure may assist with ergonomically positioning fingers of the user when the user operates endoscope 101 or other medical devices, may reduce hand strain caused by excessive movement and/or reaching of fingers when the user operates endoscope 101, and may reduce the chance of the user losing his or her grip. The handle assemblies 306, 406, 506, 606 and the actuators 300, 400, 500, 600 of this disclosure may reduce the percentage of maximal voluntary contraction (%MVC) of muscles, in particular, the extensor digitorum, brachioradialis, and the palmaris longus, during an endoscopic procedure or any medical procedure. Also, the handle assemblies 306, 406, 506, 606 and the actuators 300, 400, 500, 600 may help prevent repeated repositioning of a user's hand on a medical device handle due to fatigue, strain, or other difficulty. Each of the aforementioned handle assemblies 306, 406, 506, 606 and the actuators 300, 400, 500, 600 whether used in conjunction with an endoscope system or any other medical device, may be used to enhance and/or facilitate a user's grip on a handle. Any portion of the handle assemblies or actuators discussed herein may be incorporated into a handle of an endoscope or other medical device to improve a user's operation of the device. The handle assemblies 306, 406, 506, 606 and the actuators 300, 400, 500, 600 of this disclosure may allow multiple users with different size hands and/or fingers to comfortably use the same handle assembly.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A handle assembly for a medical device comprising:
a handle body;
a wire actuator for controlling movement of an actuation wire within the medical device, wherein the wire actuator is configured to move across an exterior surface of the handle body between a first position and a second position, and wherein the wire actuator comprises:
a contact portion including a first portion positioned exterior to the handle body and a second portion positioned within an interior portion of the handle body; and
an interior body coupled to (i) the contact portion and (ii) a shaft within the handle body;
an umbilicus; and
a button, wherein the umbilicus, the button, and the wire actuator are each disposed along a common exterior surface of the handle body, and wherein the button is disposed between the umbilicus and the wire actuator along the common exterior surface.

2. The handle assembly of claim 1, wherein a ring is rotatably coupled to the shaft via couplings that extend radially outward from a central longitudinal axis of the shaft, and wherein a rod is rotatably coupled to the ring at a first end and to the actuation wire at a second end.

3. The handle assembly of claim 1, wherein the actuation wire is coupled to an elevator at a distal end of the medical device.

4. The handle assembly of claim 2, wherein the shaft is rotatable relative to the handle body and longitudinally extends in a direction transverse to a central longitudinal axis of the medical device.

5. The handle assembly of claim 1, wherein the contact portion is configured to move along a curved path.

6. The handle assembly of claim 1, further comprising at least one knob actuator, wherein the contact portion of the wire actuator is positioned between the button and the at least one knob actuator.

7. The handle assembly of claim 1, wherein the handle body includes a curved portion between the wire actuator and the umbilicus, wherein the button projects outward from the curved portion.

8. The handle assembly of claim 1, wherein the common exterior surface includes a curved portion that leads to the umbilicus, and wherein the button projects outwardly from the curved portion between the umbilicus and the wire actuator.

9. The handle assembly of claim 6, wherein the handle body includes a recess, and wherein the recess is configured to receive the at least one knob actuator.

10. The handle assembly of claim 1, further comprising at least one knob actuator disposed on a first lateral side of the handle body, wherein the common exterior surface is disposed on a side of the handle body opposite the first lateral side, and wherein the button is configured to control an imaging device.

11. A handle assembly for a medical device comprising:
a handle body;
a wire actuator for controlling movement of an actuation wire within tht medical device, wherein the wire actuator is configured to move across an exterior surface of the handle body between a first position and a second position, and wherein the wire actuator comprises:
a contact portion including a first portion positioned exterior to the handle body and a second portion positioned within an interior portion of the handle body; and
an interior body coupled to (i) the contact portion and (ii) a shaft within the handle body;
an umbilicus; and
a button projecting outwardly from a curved portion of the handle body of the handle, wherein the curved portion leads to the umbilicus, and wherein the button is disposed between the umbilicus and the wire actuator.

12. The handle assembly of claim 11, wherein the button is spaced from a central longitudinal axis of the handle body.

13. The handle assembly of claim 11, further comprising a recess, wherein the recess is defined by a first surface transverse to a second surface, wherein each of the first surface and the second surface is on a first lateral side of the handle body, and wherein the first lateral side is on an opposite side from the umbilicus.

14. The handle assembly of claim 11, further comprising:
a knob actuator configured to rotate about a first axis,
wherein the wire actuator is an elevator actuator, wherein the elevator actuator rotates about the first axis, wherein the elevator actuator and the button are at a proximalmost end of the handle body.

15. The handle assembly of claim 11, further comprising a slot positioned at a proximalmost end of the handle body, wherein the slot is configured to receive a portion of the wire actuator.

16. A handle assembly for a medical device comprising:
a handle body;
a wire actuator for controlling movement of an actuation wire within the medical device, wherein the wire actuator is configured to move across an exterior surface of the handle body between a first position and a second position, and wherein the wire actuator comprises:
a contact portion including a first portion positioned exterior to the handle body and a second portion positioned within an interior portion of the handle body; and
an interior body coupled to (i) the contact portion and (ii) a shaft within the handle body;
an umbilicus; and
a knob actuator positioned within a recess of the handle body;
wherein the recess of the handle body is disposed between the knob actuator and the wire actuator.

17. The handle assembly of claim 16, wherein the recess is defined by a circular surface that intersects with a curved surface, wherein the curved surface is transverse to the circular surface.

18. The handle assembly of claim 17, wherein an outer circumference of the knob actuator is aligned with an edge of the curved surface.

19. The handle assembly of claim 16, further comprising a button, wherein the handle body includes a curved portion between the wire actuator and the umbilicus, and wherein the button projects outward from the curved portion.

20. The handle assembly of claim 16, further comprising a button, wherein the umbilicus, the button, and the wire actuator are each disposed along a common exterior surface of the handle body, and wherein the button is disposed between the umbilicus and the wire actuator along the common exterior surfa
